(19) United States
(12) Patent Application Publication
(10) Pub. No.: US 2020/0306313 A1
(43) Pub. Date: Oct. 1, 2020

(54) METHODS AND COMPOSITIONS FOR NON-CYTOTOXIC STEM CELL TRANSPLANTATION

(71) Applicant: THE BOARD OF REGENTS OF THE UNIVERSITY OF TEXAS SYSTEM, Austin, TX (US)

(72) Inventors: Senlin LI, San Antonio, TX (US); Robert A. CLARK, San Antonio, TX (US); Cang CHEN, San Antonio, TX (US)

(21) Appl. No.: 16/900,178

(22) Filed: Jun. 12, 2020

Related U.S. Application Data
(62) Division of application No. 15/308,915, filed on Nov. 4, 2016.
(60) Provisional application No. 61/990,698, filed on May 8, 2014.
(60) Provisional application No. 62/061,370, filed on Oct. 8, 2014.

(51) Int. Cl. A61K 35/28; A61M 1/38; C12N 5/0789; A61K 31/395; A61K 38/19; A61M 1/34

(52) U.S. Cl. CPC A61K 35/28; A61M 1/38; C12N 5/0647; C12N 2510/00; A61K 38/193; A61M 1/3496; A61M 2202/0437; A61K 31/395

(57) ABSTRACT

Certain embodiments are directed to compositions and methods for non-cytotoxic hematopoietic stem cell transplantation.

This application is a divisional of U.S. application Ser. No. 15/308,915 filed Nov. 4, 2016, which is a national phase application under 35 U.S.C. § 371 of International Application No. PCT/US2015/029612 filed May 7, 2015, which claims priority to U.S. Application Nos. 61/990,698 filed May 8, 2014 and 62/061,370 filed Oct. 8, 2014, all of which are incorporated herein by reference in their entirety.

### STATEMENT REGARDING FEDERALLY FUNDED RESEARCH

Certain embodiments of this invention were made with government support under NS046004 awarded by the National Institutes of Health. The government has certain rights in the invention.

### BACKGROUND

Hematopoietic stem cell transplantation (HCST) is used for treating a variety of blood diseases, autoimmune conditions, malignant diseases, and is being developed to treat various other diseases. During HCST, hematopoietic stem cells (HSCs) are depleted in the subject and then new HSCs are infused into the subject. Currently, subjects endure a harsh conditioning regimen consisting of cytotoxic chemotherapy and/or irradiation known as myeloablation prior to HSCT to eradicate target cells and deplete the HSCs. This treatment severely impacts immune system function and may increase a subject's risk of acquiring opportunistic infections.

Myeloablation helps prevent rejection of the transplant by the subject's immune system when the cells are from a non-autologous donor. Similar conditioning regimens are also used in autologous transplants where the subject is the donor and cells from the subject are removed and later returned to the same subject. There are some non-myeloablative conditioning regimens (though less effective) available in which lower doses of chemotherapy and/or irradiation are used that do not eradicate all of the hematopoietic cells, but subjects may suffer the same side effects seen with myeloablative regimens. There remains a need for additional methods for HSCT.

### SUMMARY

Certain embodiments of the invention provide methods for non-cytotoxic HSCT. Non-cytotoxic HSCT includes methods that do not use chemotherapy or irradiation to condition the subject prior to administration of transplant or replacement cells. In certain aspects, the HSCT methods described herein include administering a stem cell mobilization agent to stimulate migration of target stem cells out of a stem cell niche, followed by the administration of exogenous (e.g., transplant or replacement) stem cells that subsequently migrate to the appropriate stem cell niche. As used herein exogenous stem cells refers to stem cells other than those stem cells occupying the stem cell niche at the time of mobilization. Thus, exogenous stem cells include stem cells previously isolated from the same patient and returned to that same patient at a later time. In certain aspects this mobilization and transplantation cycle is performed for a number of cycles. In a further aspect the mobilization/transplantation cycle is performed at least four times.

Currently multiple cycles of stem cell transplantation is not an ideal method for human clinical use. In certain aspects, such as with a condition that results from a homozygous deficiency, a large percentage of cells will need to be replaced so that the deficiency is adequately compensated for, requiring 50, 60, 70, 80, 90%, or more of the stem cell niche to be occupied by replacement cells. In another aspect, such as a condition that result in aberrant gene dosage, such as a condition resulting from a heterozygous condition, a smaller percentage of engraftment of replacement stem cells may be needed, e.g., 20, 30, 40 up to 50% of the stem niche to be occupied by a replacement stem cells. And in a third scenario, such as a therapeutic scenario an effective amount of replacement cells may need to be in a lower percentage due to the therapeutic effect of a secreted protein or other biomolecule, e.g., 0.1, 1, 5, 10, 15, up to 20% of the stem niche to be occupied by a replacement stem cells. Thus, various conditions will require a plurality of cycles to achieve the intended effect.

As used herein, a stem cell niche is a tissue microenvironment where stem cells are found, and the microenvironment interacts with stem cells to regulate stem cell fate. The word ‘niche’ can be in reference to the in vivo stem cell microenvironment. In the body, stem cell niches maintain stem cells in a quiescent state, but after activation, the surrounding microenvironment actively signals to stem cells to promote either self-renewal or differentiation to form new cells or tissues. Several factors contribute to the characteristics within a particular niche: (i) cell-cell interactions between stem cells, and between stem cells and neighboring cells; (ii) interactions between stem cells and adhesion molecules, extracellular matrix components, growth factors, and cytokines; and (iii) the physiochemical nature of the microenvironment including oxygen tension, pH, ionic strength (e.g., Ca²⁺ concentration) and presence of various metabolites. The mobilization of the target stem cells (the movement from or evacuation of a niche) increases the probability that a transplant or replacement stem cell will occupy the stem cell niche.

The “target stem cell” is defined as an endogenous stem cell that is mobilized, collected, and/or depleted from a subject. A “transplant or replacement stem cell” is a stem cell that is being introduced to a subject. The transplant or replacement stem cell can be a therapeutic stem cell in that it has been genetically engineered, conditioned, or otherwise modified to be therapeutic to the subject. Genetic engineering refers to the direct manipulation of the genome or other nucleic acids of a cell for various effects including, but not limited to, reducing expression of a gene wherein the expression of a target protein is reduced or prevented; alterations in the level of expression (positive or negative) of a protein, for example expression of an endogenous protein in a cell type that typically does not express a target protein or an increased expression of protein that is expressed at some baseline level; and/or expression of a novel or non-endogenous protein, expression of an RNA molecule, etc. In certain aspects a cell can be engineered to produce a therapeutic protein, such as a growth factor, monoclonal antibody, enzyme, etc. Genetic engineering can include insertion of nucleic acids into the genome (chromosomal manipulation) or introduction of episomal expression vectors into the cell (extra-chromosomal manipulation).

Certain embodiments are directed to methods of non-cytotoxic stem cell transplant or replacement comprising: (a) administering at least one stem cell mobilization agent to a subject, wherein a target stem cell population migrates from a host stem cell niche into the subject's circulating blood compartment; (b) removing the mobilized target stem cells from the subject (e.g., apheresis); (c) administering transplant or replacement stem cells to the subject, wherein the transplant or replacement stem cells migrate to and occupy the host stem cell niche; and (d) repeating steps (a)-(c) 2, 3, 4, 5, 6, 7, 8, 9, or more times.

In certain aspects the transplant or replacement stem cells are therapeutic stem cells. In further aspects the therapeutic stem cells are isolated target stem cells that have been manipulated in vitro. In certain aspects the transplant, replacement, and/or therapeutic stem cells are isolated from the subject to be treated. In other aspects the transplant, replacement, and/or therapeutic stem cells are isolated from a heterologous source, i.e., a source or donor that is not the subject to be treated. The term “isolated” refers to a cell, a nucleic acid, or a polypeptide that is substantially free of heterologous cells or cellular material, bacterial material, viral material, and/or culture medium of their source of origin; or chemical precursors or other chemicals when chemically synthesized. A donor can be an autologous, allogeneic, or xenogeneic (a non-genetically identical donor of another species) donor. In certain aspects the therapeutic stem cells are genetically engineered. In certain aspects the transplant or replacement stem cells are from an autologous donor. In a further aspect the transplant or replacement stem cells are from an allogeneic donor. In a still further aspect the transplant or replacement cells are from a xenogeneic donor. In certain aspects the target stem cell is a hematopoietic stem cell. In certain aspects the transplant or replacement stem cell is a hematopoietic stem cell or a hematopoietic stem cell precursor cell.

In certain aspects a mobilization agent can be selected from interleukin-17 (IL- 17), AMD3100, granulocyte-colony stimulating factor (G-CSF), anti-sense VLA-4 receptor (e.g., ATL1102, (Antisense Therapeutics Limited)), and/or other agents known to mobilize stem cells. In certain aspects the mobilization agent is granulocyte-colony stimulating factor. In certain aspects a mobilization agent includes AMD3100. In a further embodiment the subject is administer both G-CSF and AMD3100. In a further aspect the mobilization agent can be administered prior to or during administration of the transplant or replacement stem cells to the subj ect.

In certain aspects the isolated target stem cells are manipulated by genetically modifying and/or in vitro conditioning the isolated cells from the subject.

Certain embodiments are directed to methods of treating HIV infection comprising: (a) administering at least one hematopoietic stem cell mobilization agent to a subject infected with HIV, wherein the subject's hematopoietic stem cells migrate from the hematopoietic stem cell niches to the blood; (b) removing the hematopoietic stem cells from the subject's blood; (c) administering an HIV resistant hematopoietic stem cell; and (d) repeating steps (a)-(c) four or more times. In certain aspects the HIV resistant stem cell is an engineered autologous stem cell. The method can further comprise isolating the mobilized hematopoietic stem cells from the subject and manipulating the isolated hematopoietic stem cells by genetically engineering the hematopoietic stem cell to be resistant to HIV infection. In certain aspect the cells are selected to be non-infected cells. The HIV resistant stem cell can be selected for or engineered to be a CCR5 deficient stem cell. A CCR5 deficient stem cell is a cell engineered to either not express CCR5 or express a CCR5 that does not facilitate HIV infection of the stem cell or its progeny. In certain aspects the CCR5 deficient stem cell is a CCR5 432-like stem cell, i.e., a stem cell being HIV infection resistant as is CCR 432 cells.

Certain embodiments are directed to methods for treating Parkinson's disease comprising: (a) administering at least one hematopoietic stem cell mobilization agent to a subject having Parkinson's disease, wherein the subject's hematopoietic stem cells migrate from the hematopoietic stem cell niches to the blood; (b) removing the hematopoietic stem cells from the subject's blood; (c) administering a therapeutic hematopoietic stem cell containing an expression cassette configured to express a nerve growth factor in the subject specifically when differentiated into a macrophage; and (d) repeating steps (a)-(c) five or more times. In certain aspects the therapeutic stem cell is an autologous stem cell. The method may further comprise isolating the mobilized hematopoietic stem cells from the subject; and manipulating the isolated hematopoietic stem cells by genetically engineering the hematopoietic stem cells to contain a nerve growth factor, wherein the nerve growth factor is expressed in macrophages that differentiate from the engineered hematopoietic stem cells. In certain aspects the nerve growth factor is selected from glial cell line derived neurotrophic factor (GDNF) or neurturin (NTN).

Further embodiments are directed to methods for treating Alzheimer's disease comprising: (a) administering at least one hematopoietic stem cell mobilization agent to a subject having Alzheimer's disease, wherein the subject's hematopoietic stem cells migrate from the hematopoietic stem cell niches to the blood; (b) removing the hematopoietic stem cells from the subject's blood; (c) administering a therapeutic hematopoietic stem cell containing an expression cassette configured to express brain-derived neurotrophic factor (BDNF) in the subject specifically when differentiated into a macrophage; and (d) repeating steps (a)-(c) four, five or more times. The therapeutic stem cell can be an autologous stem cell. The methods can further comprise isolating the mobilized hematopoietic stem cells from the subject; and manipulating the isolated hematopoietic stem cells by genetically engineering the hematopoietic stem cells to contain brain-derived neurotrophic factor, wherein the brain-derived neurotrophic factor is expressed in macrophages that differentiate from the engineered hematopoietic stem cells.

Still further embodiments are directed to methods for treating atherosclerosis comprising: (a) administering at least one hematopoietic stem cell mobilization agent to a subject having atherosclerosis, wherein the subject's hematopoietic stem cells migrate from the hematopoietic stem cell niches to the blood; (b) removing the hematopoietic stem cells from the subject's blood; (c) administering a therapeutic hematopoietic stem cell containing an expression cassette configured to express a nuclear receptor specifically when differentiated into a macrophage; and (d) repeating steps (a)-(c) four, five or more times. The therapeutic stem cell can be an autologous stem cell. The method can further comprise isolating the mobilized hematopoietic stem cells from the subject; and manipulating the isolated hematopoietic stem cells by genetically engineering the hematopoietic stem cells to contain apoE or LXRa, wherein the apoE or LXRa is expressed in macrophages that differentiate from the engineered hematopoietic stem cells.

The terms “individual,” “host,” “subject,” and “patient” are used interchangeably to refer to an animal that is the object of treatment, observation and/or experiment. “Animal” includes vertebrates, such as mammals. “Mammal” includes, without limitation, mice, rats, rabbits, guinea pigs, dogs, cats, sheep, goats, cows, horses, primates, such as monkeys, chimpanzees, and apes, and humans. In certain embodiments the subject is a human subject.

The terms “ameliorating,” “treating,” “treatment,” “therapeutic,” or “therapy” do not necessarily mean total cure or abolition of the disease or condition. Any alleviation of any undesired signs or symptoms of a disease or condition, to any extent, can be considered amelioration, and in some respects a treatment and/or therapy.

As used herein, the term “progenitor cells” refers to cells that, in response to certain stimuli, can form differentiated cells, such as hematopoietic or myeloid cells. As used herein, “stem” cells are less differentiated forms of progenitor cells. Typically, such cells are often positive for CD34 in humans.

The term “providing” is used according to its ordinary meaning “to supply or furnish for use.” In some embodiments, a protein is provided by administering the protein, while in other embodiments, the protein is effectively provided by administering a nucleic acid that encodes the protein or a cell that synthesizes the protein.

Other embodiments of the invention are discussed throughout this application. Any embodiment discussed with respect to one aspect of the invention applies to other aspects of the invention as well and vice versa. Each embodiment described herein is understood to be an embodiment of the invention that is applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 is a schematic of a non-cytotoxic stem cell transplant or replacement method.

FIG. 2. Human apoE transgenic expression in macrophages and reduction of atherosclerosis of apoE−/− mice.

FIG. 3. Dual luciferase analysis of synthetic promoters. Thp-1, RAW264.7, Mono Mac-1, HeLa, 293, and Caco-2 cells were transfected and luciferase activity measured 48 hours later (n=3 to 10). Synthetic promoters are indicated by clone number.

FIGS. 4A-E. Peripheral blood flow cytometry analysis of MSP-GFP mice showing GFP expression mostly in CD11b-positive cells (n=10, P<0.0001) 3 weeks after transplantation (FIG. 4A). About 7% of the CD11b-negative cells expressed very low levels of GFP (n=10, P<0.0001) (FIG. 4B). No GFP expression was observed in red blood cells from MSP GFP mice, whereas red blood cells from control mice, transplanted with bone marrow cells transduced with lentivector encoding GFP driven by ubiquitous promoter CMV, were GFP positive (FIG. 4C). GDNF levels by ELISA in the blood plasma (n=5) of MSP-GFP and MSP-GDNF mice 17 weeks after transplantation (FIG. 4D). GFP-positive cells in the peripheral leukocytes of MSP-GFP mice at various time points following bone marrow transplantation (n=7) (FIG. 4E).

FIGS. 5A-D. Total number of Iba1-and GFP-positive cells in the nigra of MSP-GFP mice assessed by stereology (FIG. 5A). Proportion of bone marrow derived (GFP-positive) microglia in the nigra of MSP-GFP mice (n=3 in each group) (FIG. 5B). Sections of the midbrain of saline and 1-methyl-4-phenyl-1,2,3,6-tetrahydropyridine (MPTP) treated MSP-GFP mice showing GFP- and Iba1 (microglia marker)-positive cells in the SNpc (FIG. 5C). Sections of MPTP treated MSP-GFP mice showing genetically modified bone-marrow derived microglia (green) in close proximity with TH-positive neurons (red) (FIG. 5D).

FIGS. 6A-B. GDNF levels by ELISA in the substantia nigra (FIG. 6A, n=5, P<0.002) striatum (FIG. 6B, n=5, P<0.001) of MSP-GFP and MSP-GDNF mice nine weeks after the last injection of MPTP.

FIGS. 7A-B. Plots of quantitative stereologic data illustrating TH-positive cells in the SNpc from mice that were continuously treated with saline (n=3) or 5 mg MPTP/kg daily (n=2) for 28 days (FIG. 7A). Semi-quantitative analysis of GFP-positive cells in the nigra of mice that were continuously treated with saline (n=3) or 5 mg MPTP/kg daily (n =2) for 28 days. Each bar represents the mean±standard error of the total number of GFP-positive cells per five representative sections of substantia nigra pars compacta per animal (FIG. 7B).

FIG. 8. Plots of quantitative stereologic data showing total number of Nissl-stained cells in the SNpc 9 weeks post MPTP treatment (***P<0.001). The number of animals in each group is shown in parentheses.

FIGS. 9A-B. Schematic representation of the lentiviral vector (LV-MSP-Tet-On-GDNF) design. GDNF expression is driven by doxycycline-regulated macrophage specific promoter (MSP). Tet-ON relies on repressors (tetR-KRAB, coded by tTR-KRAB) that in the absence of doxycycline bind to tetO and suppress the expression of GDNF as well as its own via an autoregulatory loop, whereas in the presence of doxycycline tTRKRAB does not bind tetO, thus allowing GDNF expression (FIG. 9A). Bone marrow-derived macrophages were transduced with LV-MSP-Tet-On-GDNF. Culture medium was harvested at 24 h post transduction and GDNF concentration was measured by an ELISA kit (FIG. 9B).

FIGS. 10A-H. Plots of quantitative stereologic data showing total number of TH-positive (P<0.001) neurons in the SNpc. The number of animals in each group is shown in parentheses (FIG. 10A). Images showing alpha-synuclein immunoreactive inclusions in TH-immunoreactive neurons in the SNpc of 1-methyl-4-phenyl-1,2,3,6-tetrahydropyridine/probenecid (MPTP/p) mice (FIG. 10B). Plots of quantitative data illustrating impaired motor performance by MPTP/p mice on rotarod test (P<0.001) (FIG. 10C). Total activity (FIG. 10D), and rearing behavior (FIG. 10E) assessed by open field test. MPTP/p animals crossed significantly less number of squires (a measure of total activity; P<0.001) in the open field. These animals also displayed significantly less rearing behavior (P<0.001) compared saline/p mice. Plots of quantitative data showing impaired performance on beam walking test (FIG. 10F). MPTP/p mice took significantly (P<0.001) more time to traverse a lm long, 8 mm diameter beam held at 45° angle. Similar results were also obtained for pole test (FIGS. 10G-H). MPTP/p mice took significantly more time to orient down (FIG. 10B, P<0.001) and descend (FIG. 10H, P<0.001) from a 55 cm long, 8 mm diameter pole held in the home cage. A total of 8 MPTP/p and 10 saline/p mice were used for behavioral analysis.

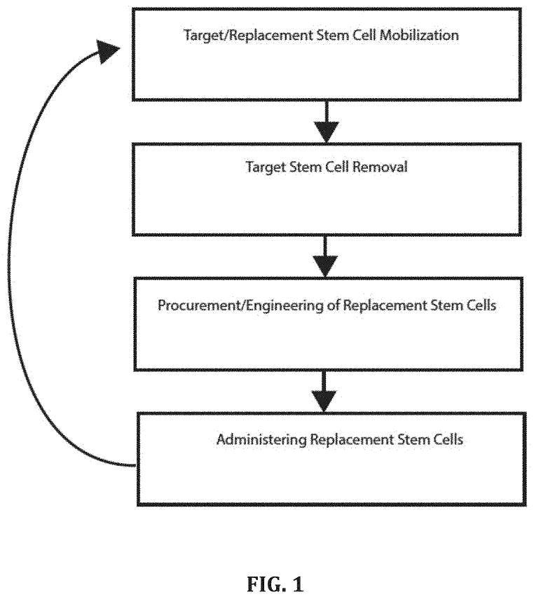

### DESCRIPTION

Hematopoietic stem cell transplantation (HSCT) is used in the treatment of a variety of hematological, autoimmune, and malignant diseases. HSCT is the transplantation of blood stem cells derived from the bone marrow (in this case known as bone marrow (BM) transplantation), blood (such as peripheral blood and umbilical cord blood), or amniotic fluid. Currently, patients endure a harsh conditioning regimen prior to HSCT known as myeloablation to eradicate the disease and hematopoietic stem cells (HSCs). “Myeloablation” refers to the severe or complete depletion of HSCs by the administration of chemotherapy and/or radiation therapy prior to HCST. This treatment severely impacts the myeloproliferative function of the hematopoietic system. Myeloablation techniques for allogeneic transplants (the transplantation of cells, tissues, or organs to a recipient from a genetically non-identical donor of the same species) can include a combination of cyclophosphamide with busulfan or total body irradiation (TBI). Autologous transplants (the transplantation of cells, tissues, or organs to a recipient from a genetically identical donor, e.g., the subject is both the recipient and the donor) may also use similar conditioning regimens. Various chemotherapy and/or radiation combinations can be used depending on the disease.

The indiscriminate destruction of HSCs can lead to a reduction in normal blood cell counts, such as lymphocytes, neutrophils, and platelets. Such a decrease in white blood cell counts also results in a loss of immune system function and increases the risk of acquiring opportunistic infections. Neutropenia resulting from chemotherapy and/or radiation therapy may occur within a few days following treatments. The subject remains vulnerable to infection until the neutrophil counts recover to within a normal range. If the reduced leukocyte count (leukopenia), neutrophil count (neutropenia), granulocyte count (granulocytopenia), and/or platelet count (thromboocytopenia) become sufficiently serious, therapy must be interrupted to allow for recovery of the white blood cell and/or platelet counts.

There are “non-myeloablative” conditioning regimens being tested using lower dose chemotherapy and/or radiation therapy that do not eradicate all of the hematopoietic cells, but the subjects still suffer similar side effects, just to a lesser degree. Notably, the treatment of non-malignant diseases by autologous HSCT does not require cytotoxic conditioning regimens. For example, current experimental non-myeloablative conditioning regimens include antibody-based (Czechowicz et al. Science. 2007, 318(5854):1296-1299; Xue et al. Blood. 2010, 116:5419-5422), type I interferon-mediated (Sato et al. Blood. 2013, 121(16):3267-3273), and G-CSF-modulated pre-transplant conditioning (Mardiney and Malech, Blood. 1996, 87(10):4049-4056; Barese et al. Stem Cells. 2007, 25(6)1578-1585). However, the antibody-mediated conditioning regimen (Czechowicz et al.) works only in immune-deficient subjects, not for HSCT recipients that are immune-competent. Type I interferon-mediated and G-CSF-modulated pre-transplant conditioning regimens still require irradiation or chemotherapy, but at reduced (non-myeloablative) doses. AMD3100 was tried without irradiation and chemotherapy and shown not to be sufficiently effective. Embodiments of methods described herein provide an effective “non-cytotoxic” regimen (i.e., a regimen with little to no cytotoxicity) so that the side effects of irradiation and chemotherapy are avoided.

### I. STEM CELL TRANSPLANTATION OR REPLACEMENT

Stem cells are undifferentiated cells that can differentiate into specialized cells and can divide (through mitosis) to produce more stem cells. In mammals, there are two broad types of stem cells: (i) embryonic stem cells, which are isolated from the inner cell mass of blastocysts, and (ii) adult stem cells, which are found in various tissues. In adult organisms, stem cells and progenitor cells act as a repair system for the body, replenishing adult tissues. Usual sources of adult stem cells in humans include bone marrow (BM), adipose tissue (lipid cells), and blood. Harvesting stem cells from blood can be done through apheresis, wherein blood is drawn from a donor (similar to a blood donation), and passed through a machine that extracts stem cells and returns other portions of the blood to the donor. Another source of stem cells is umbilical cord blood.

Adult stem cells are frequently used in medical therapies, for example in bone marrow transplantation. Stem cells can now be grown, manipulated, and/or transformed (differentiated) into specialized cell types with characteristics consistent with cells of various tissues such as muscles or nerves. Embryonic cell lines and autologous embryonic stem cells generated through therapeutic cloning have also been proposed as promising candidates for therapies.

Autologous harvesting of stem cells is one of the least risky methods of harvesting. By definition, autologous cells are obtained from one's own body, just as one may bank his or her own blood for elective surgical procedures, one may also bank stem cells. Autologous stem cell transplantation is a medical procedure in which stem cells are removed, stored, and/or reintroduced into the same person. These stored cells can then be the source for transplant or replacement stem cells in the methods described herein.

Stem cell transplants are most frequently performed with hematopoietic stem cells (HSCs). Autologous HSCT comprises the extraction of HSCs from the subject and/or freezing of the harvested HSCs. After conditioning or genetic engineering of cells isolated from the subject, the subject's HSCs are transplanted into the subject. Allogeneic HSCT involves HSC obtained from an allogeneic HSC donor. Typically the allogeneic donor has a human leukocyte antigen (HLA) type that matches the subject.

Embodiments of the non-cytotoxic methods described herein comprise mobilizing a target stem cell population (inducing the movement of the stem cells to the blood or other body fluid); removing, isolating, and/or selecting a the target stem cell population from the stem cell-enriched body fluid; administering a transplant or replacement stem cell population to a subject, wherein the transplant or replacement stem cell population localizes in the niche for the target stem cell population. In certain aspects the steps of the method are repeated a number of times. Multiple rounds of transplantation can lead to an increasing representation of the transplant or replacement stem cell population in the subject.

In certain aspects hematopoietic stem cells are mobilized from their niche in the bone marrow and replaced with a therapeutic stem cell. Hematopoietic stem cells (HSCs) are bone marrow cells with the capacity to reconstitute the entire hematopoietic system. Hematopoietic stem cells are identified by their small size, lack of lineage (lin) markers, low staining with vital dyes such as rhodamine (rhodamineDULL, also called rholo), and presence of various antigenic markers on their surface. A number of the HSC markers belong to the cluster of differentiation series, like: CD34, CD38, CD90, CD133, CD105, CD45, and also c-kit (stem cell factor receptor). The hematopoietic stem cells are negative for markers used to detect lineage commitment, and are, thus, called Lin-minus (Lin−). Blood-lineage markers include but are not limited to CD13 and CD33 for myeloid, CD71 for erythroid, CD19 for B lymphocytes, CD61 for megakaryocytes for humans; and B220 (murine CD45) for B lymphocytes, Mac-1 (CD11b/CD18) for monocytes, Gr-1 for granulocytes, Ter119 for erythroid cells, Il7Ra, CD3, CD4, CD5, CD8 for T lymphocytes, etc. in mice. Antibodies can be used to deplete the lin+ cells.

Stem cells can include a number of different cell types from a number of tissue sources. The term “induced pluripotent stem cell” (iPS cell) refers to pluripotent cells derived from mesenchymal cells (e.g., fibroblasts and liver cells) through the over-expression of one or more transcription factors. In certain aspects iPS cells are derived from fibroblasts by the over-expression of Oct4, Sox2, c-Myc, and Klf4 (Takahashi et al. Cell, 126: 663-676, 2006 for example). As used herein, “cells derived from an iPS cell” refers to cells that are either pluripotent or terminally differentiated as a result of the in vitro culturing or in vivo transplantation of iPS cells.

Neural stem cells are a subset of pluripotent cells that have partially differentiated along a neural cell pathway and express some neural markers, including for example, nestin. Neural stem cells may differentiate into neurons or glial cells (e.g., astrocytes and oligodendrocytes).

A population of cells can be depleted of cells expressing certain surface markers using a selection process that removes at least some of the cells expressing various cell surface markers. This selection process may be done by any appropriate method that preserves the viability of the cells that do not express the selection marker, including for example, fluorescence-activated cells sorting (FACS) or magnetically-activated cells sorting (MACS). Preferably, depleted populations contain less than 10%, less than 5%, less than 2.5%, less than 1%, or less than 0.1% of cells expressing the selection marker.

A. Mobilization Methods

Hematopoietic stem cells reside in specific niches in the bone marrow (BM) that control survival, proliferation, self-renewal, or differentiation. In normal individuals, the continuous trafficking of HSCs between the BM and blood compartments likely fills empty or damaged niches and contributes to the maintenance of normal hematopoiesis (Wright et al. Science. 2001, 294:1933-1936; Abkowitz et al. Blood. 2003, 102:1249-1253). It has been known for many years that egress of HSCs can be enhanced by multiple agonists known as “stem cell mobilization agents.” The hematopoietic cytokine granulocyte-colony stimulating factor (G-CSF), a glycoprotein that stimulates the bone marrow to produce granulocytes and stem cells and release them into the bloodstream, is widely used clinically to elicit HSC mobilization for BM transplantation (Lapidot and Petit. Exp. Hematol. 2002, 30:973-981; Papayannopoulou, T. Blood. 2004, 103:1580-1585). Functionally, it is a cytokine and hormone, a type of colony-stimulating factor, and is produced by a number of different tissues. In addition, AMD3100 has been shown to increase the percentage of persons that respond to the therapy and functions by antagonizing CXCR4, a chemokine receptor important for HSC homing to the BM. In certain aspects a subject is administered an agent that induces movement of a stem cell from the niche and an agent that inhibits the homing of a stem cell to the niche.

The dosages and dosage regimen in which the mobilization agents are administered will vary according to the dosage form, mode of administration, the condition being treated and particulars of the patient being treated. Accordingly, optimal therapeutic concentrations will be best determined empirically at the time and place through routine experimentation.

Certain mobilization agent(s) may be administered parenterally in the form of solutions or suspensions for intravenous or intramuscular perfusions or injections. In that case, the mobilization agent(s) are generally administered at the rate of about 10 μg to 10 mg per day per kg of body weight. Methods of administration include using solutions or suspensions containing approximately from 0.01 mg to 1 mg of active substance per ml. In certain aspects the mobilization agent(s) are administered at the rate of about 10, 20, 30, 40, 50, 60, 70, 80, 90, or 100 μg to 1, 2, 3, 4, 5, 6, 7, 8, 9, or 10 mg per day per kg of body weight.

Certain mobilization agents may be administered enterally. Orally, the mobilization agent(s) can be administered at the rate of 100 μg to 100 mg per day per kg of body weight. In certain aspects the mobilization agent(s) can be administered at the rate of about 100, 150, 200, 250, 300, 350, 400, 450, or 500 μg to about 1, 5, 10, 25, 50, 75, 100 mg per day per kg of body weight. The required dose can be administered in one or more portions. For oral administration, suitable forms are, for example, tablets, gel, aerosols, pills, dragees, syrups, suspensions, emulsions, solutions, powders and granules.

The agent(s) and/or pharmaceutical compositions disclosed herein can be administered according to various routes, typically by injection, such as local or systemic injection(s). However, other administration routes can be used as well, such as intramuscular, intravenous, intradermic, subcutaneous, etc. Furthermore, repeated injections can be performed, if needed.

For in vivo administration, active agent(s) can be added to, for example, a pharmaceutically acceptable carrier, e.g., saline and buffered saline, and administered by any of several means known in the art. Examples of administration include parenteral administration, e.g., by intravenous injection including regional perfusion through a blood vessel supplying the tissues(s) or organ(s) having the target cell(s), or by inhalation of an aerosol, subcutaneous or intramuscular injection, topical administration such as to skin wounds and lesions, direct transfection into, e.g., bone marrow cells prepared for transplantation and subsequent transplantation into the subject, and direct transfection into an organ that is subsequently transplanted into the subject. Further administration methods include oral administration, particularly when the active agent is encapsulated.

B. Isolation Methods

In contrast to difficult bone marrow transplants, HSCs can be easily collected from the peripheral blood and this method provides a bigger graft, does not require that the donor be subjected to general anesthesia to collect the graft, results in a shorter time to engraftment, and may provide for a lower long-term relapse rate. In order to harvest HSCs from the circulating peripheral blood, subjects are administered one or more mobilization agents that induce cells to leave the bone marrow and circulate in the blood vessels. The subjects then undergo apheresis to enrich and collect the HSCs and then return the HSC-depleted blood to the subjects.

C. Administration Methods

The compositions can be administered using conventional modes of delivery including, but not limited to, intravenous, intraperitoneal, oral, intralymphatic, subcutaneous, intraarterial, intramuscular, intrapleural, intrathecal, and by perfusion through a regional catheter. When administering the compositions by injection, the administration may be by continuous infusion or by single or multiple boluses. For parenteral administration, the stem cell mobilization agents may be administered in a pyrogen-free, parenterally acceptable aqueous solution comprising the desired stem cell mobilization agents in a pharmaceutically acceptable vehicle. A particularly suitable vehicle for parenteral injection is sterile distilled water in which one or more stem cell mobilization agents are formulated as a sterile, isotonic solution, properly preserved.

### II. THERAPEUTIC METHODS

The methods described herein provide gentle and low-risk, but high-level, replacement of endogenous stem cells with either genetically engineered or pharmacologically rejuvenated HSCs or the combination. This HSCT strategy can translate into transformative approaches that enhance and broaden HSCT applications in clinical research and patient management, particularly for aging-associated diseases.

Ex vivo bone marrow cells may be cultured and (i) expanded to increase the population of hematopoietic progenitor cells, (ii) genetically engineered and/or (iii) otherwise conditioned, prior to reintroduction of such cells into a patient. These hematopoietic stem cells or precursor cells may be used for ex vivo gene therapy, whereby the cells may be transformed in vitro prior to reintroduction of the transformed cells into the patient. In gene therapy, using conventional recombinant DNA techniques, a selected nucleic acid, such as a gene, may be isolated, placed into a vector, such as a viral vector, and the vector transfected into a hematopoietic cell, to transform the cell, and the cell may in turn express the product encoded by the gene. The cell then may then be introduced into a patient (Wilson et al. PNAS. 1998, 85:3014-3018). However, there have been problems with efficient hematopoietic stem cell transfection (Miller. Blood. 1990, 76:271-278). A transformed cell can be engineered to express and/or secrete a therapeutic protein such as a growth factor, cytokine, monoclonal antibody (positive modulator of another proein or cell or a negative modulator of another protein or cell), ligand, enzyme, receptor, etc.

Ex vivo administration of active agents can be done by any standard method that would maintain viability of the cells, such as by adding it to culture medium (appropriate for the target cells) and adding this medium directly to the cells. As is known in the art, any medium used in this method can be aqueous and non-toxic so as not to render the cells non-viable. In addition, it can contain standard nutrients for maintaining viability of cells, if desired.

A. Methods for Treating Parkinson's Disease

Parkinson's disease (PD) is a degenerative disorder of the central nervous system characterized by shaking, rigidity, slowness of movement and difficulty with walking and gait. The motor symptoms of PD result from the death of dopamine-generating cells in the substantia nigra, a region of the midbrain; the cause of this cell death is unknown. However, mouse models of PD have shown the expression of either of the neural growth factors glial cell line-derived neurotrophic factor (GDNF) or neurturin (NTN) provide a protective effect against dopaminergic neurodegeneration (Biju et al. Molecular Therapy. 2010, 18:1536-1544; Biju et al. Neuroscience Letters 2013, 535:24-29). In clinical application, HSCs will be collected from a patient with Parkinson's disease and stored. The HSCs can be engineered to express GDNF or NTN and then transplanted back into the same subject (Biju et al., 2010). The transplantations will be repeated multiple times to get sufficient numbers of blood cells expressing GDNF or NTN.

A cell-based, non-invasive approach to treating Parkinson's disease (PD) with a neurotrophic factor can be used for protection of the dopamine (DA) neurons affected in PD. In preclinical studies, both symptomatic and neuroprotective benefits of GDNF have been demonstrated. However, GDNF crosses the blood-brain barrier (BBB) so poorly that systemic delivery is ineffective. Clinical trials involving invasive brain injection of either GDNF protein or GDNF-expressing viral vectors have shown inconsistent results. This may be at least partially attributable to insufficient delivery of this trophic factor to the degenerating nigrostriatal DA neurons due to its limited diffusion in brain tissue, as well as the large (relative to experimental rodents) target volume of the human brain. Furthermore, the chronic progressive nature of PD necessitates sustained infusion of GDNF over months/years in order to maintain DA neuron survival and function. Hematopoietic stem cell (HSC) transplantation-based macrophage/microglia-mediated GDNF delivery can be used as an additional method of treatment for PD.

This approach takes advantage of the well-known macrophage property of homing to degenerating central nervous system sites in proximity to damaged neurons, incorporates macrophage-specific synthetic promoters (MSP), and capitalizes on the long-standing clinical experience with HSC transplantation (HSCT), as well as recent advances in HSC gene therapy. The clinical scenario of this therapy is that autologous HSCs are mobilized from bone marrow, isolated from peripheral blood by apheresis, and then transduced ex vivo with an expression vector (e.g., lentiviral vector) carrying the GDNF gene. The transduced HSCs are infused into the patient after pre-conditioning, resulting in engraftment of the transplanted HSCs that will form various blood cell lineages. The therapeutic gene is expressed at high levels only in cells of the monocyte/macrophage lineage because it is under MSP control. The macrophages will infiltrate the brain and become microglial cells, which accumulate in the nigrostriatal system where neurodegeneration is focused in PD patients. These microglial cells will secret GDNF protein and make the trophic factor accessible to surrounding neurons that are affected in the patients. Indeed, similar approaches are curative for leukodystrophies, a group of rare hereditary neurodegenerative diseases.

B. Methods for Treating Atherosclerosis

Atherosclerosis, which underlies myocardial infarction, stroke, and peripheral occlusive vascular disease, is the leading cause of mortality and morbidity in the United States and other developed countries. Current therapies are generally directed at lowering LDL cholesterol levels using the statin class of drugs. The methods described herein can be used with genetically engineered macrophages to provide an additional treatment for atherosclerosis.

Macrophages, differentiated from monocytes originated from bone marrow hematopoietic stem cells (HSC), are a major player in atherogenesis. When expressed in macrophages, some genes are anti-atherogenic, whereas others are pro-atherogenic. For example, apoE expression in macrophages is anti-atherogenic or atheroprotective. As monocytes/macrophages are generally short-lived, any anti-atherogenic effects of direct genetic manipulation of them will not likely be long lasting. On the other hand, the HSCs from which macrophages originate are self perpetuating and long-lived.

Lentiviral HSC gene therapy has been studied for the amelioration of atherosclerosis. The HSCT procedure described herein can be used to express apoE in macrophages for the mitigation of atherosclerosis. The methods can further comprise isolating the mobilized hematopoietic stem cells from the subject; and manipulating the isolated hematopoietic stem cells by genetically engineering the hematopoietic stem cell to contain apoE or LXRa, wherein the apoE or LXRa is expressed in macrophages.

C. Rejuvenation Methods

Currently there are more than 39 million Americans aged 65 or older. Breakthroughs in biomedical research aiming to increase healthspan and lifespan will create economic benefit and dramatically improve the quality of life for these elderly individuals, as well as to society as a whole.

The field of aging research has now moved into developing interventions that enhance healthspan and lifespan in experimental animals. Novel pharmacologic, biological, and genetic interventions have potential to extend lifespan, delay cancers, dementias, and possibly other age-related diseases. However, these interventions have many caveats and limitations. For example, rapamycin has been shown to extend lifespans as well as healthspan in mice, but the mechanism accounting for these effects remains elusive and a growing list of side effects raises some doubts as to whether this drug will be beneficial in man.

Methods described herein can be used to extend healthspan and lifespan by rejuvenation of blood cells. Blood cells, all derived from hematopoietic stem cells (HSCs), are responsible for constant maintenance and immune protection of every cell type of the body. Age-related declines in HSCs and their progeny blood cells contribute to poor tissue oxygenation, impaired hemostasis, and decreased immune protection, as well as increased chronic inflammation and tumorigenesis (two common health problems in the elderly), which may eventually lead to ailments and deaths. The rejuvenation of blood cells can be achieved using hematopoietic stem cell transplantation (HSCT) as described herein.

The ability to replace HSCs using the methods described herein is the basis for the development of a mobilization-based conditioning regimen. Data in inbred mouse models showed ˜65% transplantation efficiency after multiple repetitions of this procedure. These methods can be used to introduce younger or rejuvenated stem cells into a subject.

The rejuvenation of blood cells can lead to healthspan and lifespan extension. A mouse model can be used that replaces old HSCs with young ones. For example, rejuvenation of blood cells by replacement for healthspan extension can be demonstrated using 20 female and 20 male C57BL/6 mice at 19 months of age that are transplanted with either age-matched old HSCs (control) or young HSCs (derived from 10-week old) by the methods described herein. Health assessments are done monthly by measurement of motor and cognitive functions using 50-hour home cage activity, stride length, grip strength, Y-maze, and novel object tests. Transplantation efficiency of 80-90% and blood cell rejuvenation is verified by characterization of blood cells at 26 and 32 months of age. In a second part of the study 36 female and 44 male C57BL/6 mice at 19 months of age are transplanted as above. Animal survival is monitored and recorded. End of life pathology is performed.

In humans, this intervention may be applied in a couple of scenarios: (1) PBSCs are collected from young adults by apheresis after s.c. injections of G-CSF and/or other HSC mobilizer(s)(e.g., G-CSF (NEUPOGEN®) and AMD3100 (MOZOBIL™)) and then cryopreserved, as currently practiced in clinic. This process is repeated multiple times (twice a year, for instance) so sufficiently large numbers of cells are stored. Once these individuals have aged, their old-phenotype blood cells would be replaced and repopulated by the young PBSCs that were obtained and stored when they were young. The replacement could reach ˜90% through repeated mobilization conditioning-based transplantations of the young PBSCs. The technology and reagents are readily applicable in today's clinic. (2) Alternatively, multiple batches of PBSCs could be collected from the elderly and cryopreserved. The HSCs from these PBSCs could be rejuvenated in vitro by genetic (over-expression of Sirt3) or by pharmacologic manipulation (treatment with cdc42 inhibitors) and transplanted back into the same individuals using the conditioning regimen and transplant method described. The HSCs can be treated ex vivo in culture with cdc42 inhibitor (CASIN) for 8-16 hours and then transplanted back to the same subjects (Florian et al., 2012) or genetically engineered to over-express SirT3 (Brown et al., 2013). (3) Another potential source of youthful HSCs would be autologous reprogramed pluripotent stem cells (such as iPS cells). Skin or blood cells can be collected from elderly patients and converted to induced pluripotent cells (iPS). The iPS cells are differentiated into HSCs, which are transplanted into the same subject (Hanna et al., 2007). The transplantation is done repeatedly to achieve sufficient replacement of HSCs.

D. Methods for Treating Alzheimer's Disease

Alzheimer' s disease (AD) is the most common form of dementia with more than 28 million affected people worldwide. Although the cause and progression of AD are not well understood, alterations in the distribution of different neurotrophic factors and in the expression of their receptors such as the brain-derived neurotrophic factor (BDNF) have been described (Tapia-Arancibia et al. Brain Research Reviews. 2008, 59(1):201-220; Schindowski et al. Genes, Brain and Behavior. 2008, 7(Supp 1):43-56) In addition, the expression of BDNF has been shown to provide a neuroprotective effect in rodent and primate models of AD (Nagahara et al. Nat. Med. 15:331-337).

E. Methods for Treating HIV Infection

Hematopoietic stem cell transplantation (HCST) can be used for treating a variety of blood diseases, autoimmune conditions, malignant diseases, and various other diseases. In some instances patients have been cured by HSCT. In the famous Berlin patient (a HIV infected leukemia patient), HSCT is credited for curing his HIV infection by replacement of his HSCs with donor HSCs homozygous for the CCR5 Δ32 mutation, which conveys cellular resistance to HIV entry and infection (Hutter et al. N Engl J Med (2009) 360(7):692-98).

Conventional HSCT using pre-conditioning with irradiation and/or chemotherapy, although an effective and life-saving treatment for patients with hematologic malignancy, is considered to be highly risky and often leads to severe infection, graft-versus-host disease, and other adverse effects. In contrast to current HSCT methodology, aspects of the methods described herein will work in all HSCT patients (both immune-deficient and immune-competent), because certain aspects are irradiation- and chemo-independent and free of the adverse effects of these conditioning regimes. In combination with cellular engineering, such as RNA-guided genome editing, the currently described HSCT method can be used to treat or cure HIV infection.

HSCT has been an important medical procedure for four decades and better conditioning regimens are constantly and actively sought by numerous physicians and investigators world-wide. Since 1993 G-CSF has been used to mobilize HSC into peripheral blood for collection, but has not been used or developed as an effective and non-toxic conditioning regimen. Current pre-transplant conditioning regimens are harsh and toxic and very detrimental to patients with non-malignant diseases (unlike patients with malignant disease, in whom toxicity can be justified because of the need to kill cancer cells). The gentle and non-toxic conditioning regimen described herein can be used advantageously with HIV infected patients. In certain aspects HSCT is used to replace endogenous HSCs with HSCs of interest and thus repopulate blood cells possessing desirable properties, particularly when combined with gene therapy approaches (Kiem et al. Mol Ther (2014) July;22(7):1235-38).

HIV resistant cells are known to exist, for example the CCR5 432 (32 base pair deletion comprising deletion of nucleotides 794 to 825 of the cDNA (GenBank accession number NM_000579.3) resulting in a frameshift and expression of a non-functional CCR5 protein) cells of the Berlin patient. CCR5 is the C-C chemokine receptor type 5, also known as CD195 and is a protein on the surface of white blood cells that is involved in the immune system as it acts as a receptor for chemokines. Many forms of HIV use CCR5 to enter and infect host cells. A few individuals carrying a CCR5 Δ32 variant in the CCR5 gene are protected against infection with HIV. The wild-type amino acid sequence of CCR5 is MDYQVSSPIYDINYYTSEPCQKINVKQIAARLLPPLYSLVFIFGFVGNMLVILILINCKR LKSMTDIYLLNLAISDLFFLLTVPFWAHYAAAQWDFGNTMCQLLTGLYFIGFFSGIFF IILLTIDRYLAVVHAVFALKARTVTFGVVTSVITWVVAVFASLPGIIFTRSQKEGLHYT CSSHFPYSQYQFWKNFQTLKIVILGLVLPLLVMVICYSGILKTLLRCRNEKKRHRAVR LIFTIMIVYFLFWAPYNIVLLLNTFQEFFGLNNCSSSNRLDQAMQVTETLGMTHCCIN PIIYAFVGEKFRNYLLVFFQKHIAKRFCKCCSIFQQEAPERASSVYTRSTGEQEISVGL (SEQ ID NO:1). The amino acid sequence of CCR5 32 is MDYQVSSPIYDINYYTSEPCQKINVKQIAARLLPPLYSLVFIFGFVGNMLVILILINCKR LKSMTDIYLLNLAISDLFFLLTVPFWAHYAAAQWDFGNTMCQLLTGLYFIGFFSGIFF IILLTIDRYLAVVHAVFALKARTVTFGVVTSVITWVVAVFASLPGIIFTRSQKEGLHYT CSSHFPYIKDSHLGAGPAAACHGHLLLGNPKNSASVSK (SEQ ID NO:2).

Since CCR5 Δ32 homozygous individuals are not common and finding an HLA-matched donor is very rare, investigators are genetically engineering HSCs to render them HIV resistant. In certain aspects a CCR5-defective HSCs can be used as donor cells to replace endogenous CCR5-normal HSCs in HSCT (Li et al. Mol Ther (2013) 21(6):1259-69; Tebas et al. New England Journal of Medicine (2014) 370(10): 901-10; Kay and Walker, New England Journal of Medicine 370(10):968-69; Kalomoiris et al., Hum Gene Ther Methods (2012) 23(6):366-75; Holt et al., Nat Biotech (2010) 3-7). The HSCT methods described herein can be used in combination with genetically engineered HIV-resistant cells or precursors thereof to treat HIV-infected individuals by reducing or eliminating HIV reservoirs in a patient. In certain aspects a treatment or cure for HIV infection can be formulated by using the HSCT methods described herein in combination with HIV-resistant hematopoietic stem cells, HIV-resistant cell precursors, and their HIV-resistant progeny. In certain aspects the HIV-resistant cell or precursor cell is a CCR5 knockout HSC.

The rationale for such a treatment CCR5-defective cells is that to infect host cells, HIV needs CCR5 as co-receptor, in addition to the CD4 molecule. People homozygous for CCR5 Δ32 mutation do not become infected by HIV (i.e., they are HIV-resistant like the Berlin Patient). In contrast, HIV can re-emerge in the drug ‘cured’ patients and in lymphoma patients receiving HSC transplants. Furthermore, because the available effective cocktail drug treatment for HIV/AIDS has to be maintained for the life of a patient (although smaller HIV-1 reservoirs are associated with reduced pathologic sequelae, such as inflammation) and the high risk associated with conventional HSCT preconditioning (irradiation and/or chemotherapy), HSCT will not likely receive IRB approval for HIV-infected patients because of the toxic conditioning steps involved, except for the rare individuals that have other indications for HSCT, such as leukemia.

It is exceedingly unlikely statistically to find an HLA-matched and CCR5 Δ32 homozygous donor. The HSCT method described herein can use autologous cells, is non-cytotoxic (totally irradiation and chemotherapy independent), is non-immunosuppressive, and can readily be performed in outpatient settings. Therefore, this method would be an ideal HSCT approach for HIV/AIDS patients.

Various approaches are known for producing an HIV-resistant cell. For example U.S. Pat. No. 8,728,458, which is incorporated herein by reference in its entirety, describes Lentiviral-based gene knockdown of CCR5. In U.S. Patent publication 2005/0220772, which is incorporated herein by reference in its entirety, donors are screened for naturally occurring stem cells to be transplanted using conventional techniques into HIV infected subjects. In another example, U.S. Patent publication 2011/0262406, which is incorporated herein by reference in its entirety, describes cells genetically engineered to be HIV-resistant. HIV-resistant cells and method of producing such are known in the art and can be used in conjunction with the current HSCT methodology for the treatment of HIV infection.

In one particular embodiment a cell rendered HIV-resistant using genome editing can be used in conjunction with the currently described HSCT method for the treatment of HIV infection. The CRISPR/Cas9 technology or other advanced similar technology can be used to generate autologous CCR5-deficient HSCs. In certain aspects integration-deficient lentiviral vectors (IDLVs) expressing guide RNA (gRNA) and Cas9 nuclease/nickase are used to infect HSCs (CD34+) isolated from the patient to be treated. In certain aspects the HSCs are isolated by apheresis. The gRNA is designed to bind to both a specific genomic DNA sequence within the CCR5 gene and to the Cas9 nuclease/nickase. Cas9 nuclease/nickase cuts the DNA at a selected site in DNA, which will be altered (mutated) during the natural DNA repair response. The mutation efficiency can reach 30% or more (measured by surveyor nuclease assay (Guschin et al., Methods Mol Biol (2010) 649:247-56) or deep sequencing). IDLV will not integrate into the host genome. Selection markers such as GFP or CD25 can be used to enrich for engineered HSCs. The CCR5-mutated HSCs are transplanted into the patient using the novel HSCT methods described herein. In certain aspects the transplantation will be repeated multiple times to reach a sufficiently high engraftment level (measured by surveyor nuclease assay or pyrosequencing) to treat or cure HIV infection of the patient. In a further aspect multiple batches of CD34+ HSCs can be collected by apheresis before the initiation of the treatment.

### III. KITS AND FORMULATIONS

In certain embodiments, the invention also provides compositions comprising 1, 2, 3 or more stem cell mobilization agents with one or more of the following: a pharmaceutically acceptable diluent; a carrier; a solubilizer; an emulsifier; a preservative; and/or an adjuvant. Such compositions may contain an effective amount of at least one stem cell mobilization agent. Thus, the use of one or more stem cell mobilization agent(s) that are provided herein in the preparation of a pharmaceutical composition of a medicament is also included.

The stem cell mobilization agents may be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The preferred form depends upon the mode of administration and the particular stem cell targeted. The compositions also preferably include pharmaceutically acceptable vehicles, carriers, or adjuvants, well known in the art.

Acceptable formulation components for pharmaceutical preparations are nontoxic to recipients at the dosages and concentrations employed. In addition to the agents that are provided, compositions may contain components for modifying, maintaining, or preserving, for example, the pH, osmolarity, viscosity, clarity, color, isotonicity, odor, sterility, stability, rate of dissolution or release, adsorption, or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18 th Ed., (A. R. Gennaro, ed.), 1990, Mack Publishing Company), hereby incorporated by reference.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

The pharmaceutical composition to be used for in vivo administration is typically sterile. Sterilization may be accomplished by filtration through sterile filtration membranes. If the composition is lyophilized, sterilization may be conducted either prior to or following lyophilization and reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

If desired, stabilizers that are conventionally employed in pharmaceutical compositions, such as sucrose, trehalose, or glycine, may be used. Typically, such stabilizers will be added in minor amounts ranging from, for example, about 0.1% to about 0.5% (w/v). Surfactant stabilizers, such as TWEEN®-20 or TWEEN®-80 (ICI Americas, Inc., Bridgewater, N.J., USA), may also be added in conventional amounts.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents. Compositions for parental administration are also sterile, substantially isotonic and made under GMP conditions.

For the compounds of the present invention, alone or as part of a pharmaceutical composition, such doses are between about 0.001 mg/kg and 1 mg/kg body weight, preferably between about 1 and 100 μg/kg body weight, most preferably between 1 and 10 μg/kg body weight.

Therapeutically effective doses will be easily determined by one of skill in the art and will depend on the severity and course of the disease, the patient's health and response to treatment, the patient's age, weight, height, sex, previous medical history and the judgment of the treating physician.

### IV. EXAMPLES

The following examples, as well as the figures, are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

### Non-Cytotoxic HSCT

Methods have been developed that provide a conditioning regimen that is gentle and substantially free of side effects. Bone marrow is the home of hematopoietic stem cells (HSCs) that are located in specialized niches. A majority of HSCs stay in the niches, but some (1-5%) leave their niche and enter and travel in the blood. The egress of HSCs from bone marrow creates empty niches that are ready to host in-coming HSCs. The egress of HSCs can be dramatically increased in the clinic by mobilization using G-CSF or a combination of G-CSF and AMD3100. This leads to increased numbers of HSCs in the peripheral blood and increased empty niches in the bone marrow. The former result is the basis for collection of HSCs from peripheral blood vessels; the latter result is the basis for the mobilization-based conditioning regimen described herein. When the empty niches reach the peak in number, the mobilized HSCs in the blood will be removed by aphresis (and processed for storage for future application). A sufficient number of transplant or replacement HSCs is administered by conventional i.v. injection/infusion and will compete with remaining endogenous circulating HSCs to occupy the available niches in the bone marrow. Indeed, data in mouse models showed up to 90% transplantation efficiency after multiple cycles of this procedure, as measured for green fluorescent protein positive (GFP+) peripheral blood cells (on the normal GFP- background).

Male C57BL/6J inbred mice at age of 14 weeks were used as recipients. G-CSF was administered to each mouse at a dose of 125 μg/kg body weight through a 0.1 ml intra-peritoneal injection every 12 hours for 4 consecutive days. AMD3100 (Mozobil) was then administered to each mouse at a dose of 5 mg/kg body weight through a 0.05 ml subcutaneous injection 14 hours after the last dose of G-CSF and 1 hour prior to bone marrow transplantation by tail-vein injection. The bone marrow cells (BMCs) were harvested from the tibias, femurs, humeri, and hip bones of GFP transgenic C57BL/6J mice by flushing with Iscove's Modified Dulbecco's Medium containing 0.5% heparin. After red blood cell lysis, either total (25×10⁶) or Scal+(7×10⁶) BMCs were given in 0.2 ml PBS containing 2% FBS to the G-CSF- and AMD3100-treated recipient mice. The Sca-1+cells were isolated by an Anti-Sca-1 MicroBead kit (Miltenyi Biotec Inc.). The whole procedure was repeated every two weeks. To assess the replacement efficacy, peripheral blood was collected and percentages of GFP+ cells were determined by flow cytometry and/or immunofluorescence microscopy. Experimental data on engraftment are compared with model-based estimates (see Table 1).

Theoretically the efficiency of transplantation can be modelled as below.
n=transplantation repeats; a=replacement rate/cycle; a′=niche emptying rate; y=ratio of donor HSCs to total HSCs (i.e., donor cells plus endogenous cleared cells); x=replacement result (cumulative % engraftment);
Based on HSCs and their niche equilibrium described above, we have;
x=1−(1−a′y)ⁿ−[1−(1−a′y)ⁿ⁻¹]*a′*(1−y)=1−(1−a)ⁿ−[1−(1−a)ⁿ⁻¹]*a′*(1−y)
When y>0.9, we can neglect the small value of the term [1−(1−a′y)n⁻¹]*a′*(1−y) and have the following:
x=1−(1−a)ⁿ
   Assuming that transplantation rate/each is 0.17 (17.0%, based on our preliminary data and the literature), then:

TABLE 1
MOBILIZATION-AIDED HSC TRANSPLANTATION

| Transplantation | Replacement result (x) (%) | | |
|---|---|---|---|
| repeats (n) | Calculated | Experimental | Adjusted |
| 1 | 17.0 | 19.21 | 22.08 |
| 2 | 31.11 | 31.42 | 36.11 |
| 3 | 42.82 | 32.91, 32.54, 35.51 | 37.83, 38.55, 40.82 |
| 4 | 52.54 | | |
| 5 | 60.61 | | |
| 6 | 67.31 | 62.27, 63.03, 64.32 | 71.58, 72.45, 73.93 |
| 7 | 72.86 | 68.47, 70.87, 80.97 | 78.70, 81.46, 93.07 |
| 8 | 77.48 | | |

Experimental x is the percentage of GFP+ cells in the blood after indicated cycles of HSCT from GFP+ to WT mice. Adjusted x was calculated based on the finding that 87% of the white blood cells are GFP+ in donor GFP transgenic mice.

Because C57BL/6J mice are highly inbred, they are genetically identical to each other. Tissue or organ transplants among them are immunologically equivalent to that in humans between homozygotic twins or with autologous transplantation and thus do not cause immune reactions, such as graft rejection or graft vs. host effects. Also, as mice are quite small in body size and have a small volume of blood, the apheresis procedure is not suitable for them. Therefore, mice were sacrificed for bone marrow harvest as a source for donor cells. In humans, the donor cells can come from his/herself after G-CSF and AMD3100 mobilization as currently practiced in the clinic. The collected cells will be cryopreserved. Multiple rounds of collection and storage will be required for later-on transplantation.

### Example 2

### Ameliorate Atherosclerosis by Overexpression of Apoe in Monocytes/macrophages

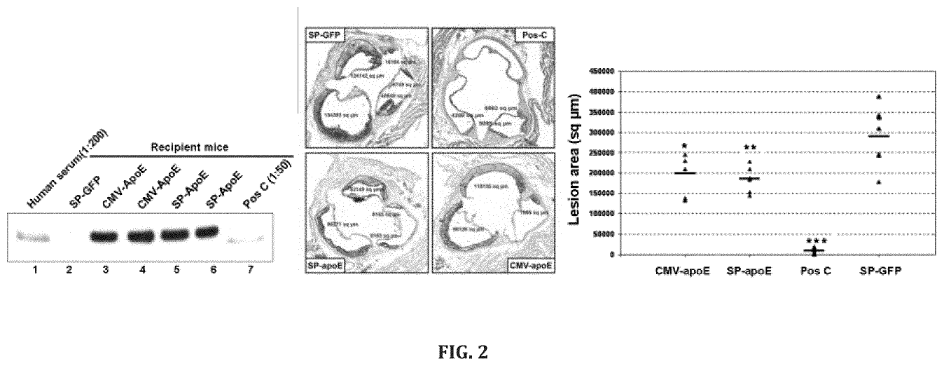

Lentiviral HSC gene therapy-based macrophage expression of human apoE reduces atherosclerotic lesions in apoE−/− mice. ApoE−/− HSC-enriched bone marrow cells transduced with the lentiviral vector encoding human apoE were used to transplant lethally-irradiated apoE−/− mice. The apoE expression was driven by a synthetic macrophage promoter (SP-apoE) developed previously. Peritoneal macrophages collected from recipient mice 16 weeks post-transplant were shown to express human apoE at high levels (FIG. 2, left panel). Macrophage expression of apoE from 10 to 26 weeks of age significantly reduced atherosclerotic lesions in recipient apoE−/− mice (FIG. 2, middle and right panels). In FIG. 2, SP-GFP, SP-apoE, and CMV-apoE (CMV promoter driving human apoE gene) were the lentiviral vectors used in these transduction and transplantation experiments, while Pos C indicated wild-type bone marrow donor group (He et al., Hum. Gene Ther. 17(9), 949 (2006)).

### EXAMPLE 3

### Treatment of Parkinson's Disease by Protection of Nigrostriatal Dopamine Neurons Through Macrophage/Microglia Delivery of Growth Factors

The MitoPark™ mouse model provides an incisive means for addressing the limitations of other mouse models of Parkinson's disease. The MitoPark™ mouse represents a conditional knockout of mitochondrial transcription factor A (Tfam) in DA neurons. The TFAM protein promotes mtDNA transcription and replication. Although human genetic mutations in Tfam have not yet been linked to PD, sporadic PD is characterized by mitochondrial dysfunction and a role for mitochondria in PD pathogenesis is widely accepted. MitoPark™ mice were noted to possess several characteristics of human PD and to be an especially faithful model of PD in comparison with most currently available murine models. The chronic and progressive nature of DA neuron loss will not only complement previous studies of MPTP-induced acute loss of DA neurons, but will also allow the inventors to intervene in either therapeutic or preventive paradigms.

MitoPark™ mice exhibit progressive impairment in spontaneous locomotor activity, evident from 10-12 weeks of age. Vertical movements declined earlier and faster than horizontal movements (data not shown), modeling the early occurrence of axial postural instability in PD. Locomotor deficits were transiently reversed by administration of L-DOPA. In addition, MitoPark™ mice were found to developed impairments in rotarod performance. Interestingly, sucrose preference tests showed apparent depressive symptoms. The MitoPark™ mice began to lose weight from ˜20 weeks and died at 29-33 weeks of age, at which point the majority of substantia nigra DA neurons had been lost. Thus, the MitoPark™ mice exhibit PD-like phenotypes that are consistent with the reports in the literature.

To assess macrophage brain infiltration, 14 week-old MitoPark™ mice and littermate controls were transplanted with GFP+ bone marrow cells from donor GFP transgenic mice of the same age. Conditioning for transplant was accomplished by head-protected irradiation (to avoid a potential contribution of brain irradiation-induced macrophage infiltration) using a customized lead tube. Engraftment efficiency was ˜80% as evidenced by the percentage of GFP+ cells in the peripheral blood of the recipients. Five weeks post-transplant, the mice were sacrificed to evaluate macrophage homing to SN. In control littermates, few GFP+ cells were observed in the SN, whereas numerous GFP-expressing cells, most of which were also positive for the microglial marker Ibal, were found in the SN of MitoPark™ mice.

HSC-based macrophage delivery of GDNF can be used to protect the nigrostriatal dopaminergic system, leading to significant amelioration of the pathologic changes, biochemical alterations, and neurologic defects without major adverse effects. Bone marrow cells enriched for HSCs from syngeneic donor mice at 12, 18, and 24 weeks of age are transduced with lentiviral vectors expressing hGDNF or GFP cDNA driven by a macrophage-specific promoter (MSP-GDNF or MSP-GFP). MSP-GFP-2A-GDNF lentivectors are also used in some studies. Transduced cells are transplanted into head-protected irradiated (to mitigate any concern that direct brain irradiation might cause BBB disruption, thereby facilitating macrophage infiltration) MitoPark™ mice of the same ages. Of note, irradiation as a conditioning method is most convenient and widely used in mice, but the clinical phases in PD patients uses the methods described herein, not irradiation. Transduction/transplantation efficiency is confirmed 4 weeks post-HSCT. Body weight, behavioral tests, tissue collection, various examinations, and data analysis is performed.

To achieve success with bone marrow hematopoietic stem cell-derived macrophage-mediated GDNF gene therapy, a series of powerful macrophage-specific synthetic promoters (MSP) were designed that restrict transgene expression to this lineage (He et al., 2006). Lentivirus-transduced bone marrow stem cell-derived macrophages showed strong and stable transgene expression under this promoter for up to 15 months (the longest time point studied) after transplantation. Using a highly active MSP, the utility of genetically modified bone marrow stem cell-derived macrophages was tested as a vehicle to deliver GDNF to the site of neurodegeneration in a mouse model of PD. It was shown that macrophage-mediated GDNF treatment dramatically ameliorated MPTP-induced degeneration of dopaminergic neurons in substantia nigra and its terminals in the striatum, stimulated axon regeneration, and reversed hypoactivity.

Although the approach described herein is superior to existing methods of GDNF delivery, enhancement of TH-immunoreactivity, DA metabolism, and behavioral change in this study was similar to the several previous studies. The MPTP-alone regimen used in proof-of-principle experiments resulted in only modest reduction (approximately 50%) in TH-positive cells. Moreover, spontaneous recovery of the nigrostriatal system that is typically observed with this MPTP regimen puts limits on assessing motor coordination. To overcome these limitations and show the superiority of bone marrow-derived macrophages mediated GDNF delivery, the inventors propose the use a chronic MPTP/probenecid mouse model. In this model, dopamine cell loss is progressive and exceeds 70%, extracellular glutamate is elevated, Lewy body-like cytoplasmic inclusions are formed and inflammation is chromic (Meredith et al., 2008). Furthermore, the behavioral impairment persists for up to 6 months post-MPTP/p treatment. The inventors have standardized this model in their lab. In previous work, GDNF treatment was started before the initiation of neurodegeneration, which would never be the case in the clinic as fifty percent of the neurons are already lost before detectable clinical symptoms appears. The inventors initiate GDNF treatment after considerable neurodegeneration has occurred. Toward this end a tetracycline-regulated lentiviral vector expressing human GDNF gene driven by MSP (LV-MSP-Tet-On-GDNF) has been developed. This vector allows one to “Switch-ON” GDNF expression at various time points after neurodegeneration has occurred, thereby closely mimicking early, middle, and late stages of clinical parkinsonism. Macrophage cell line RAW 264.7 and bone marrow-derived macrophages transduced with the LV-MSP-Tet-On-GDNF vector showed robust expression of GDNF after treatment with doxycycline, a member of the tetracycline family of antibiotics.

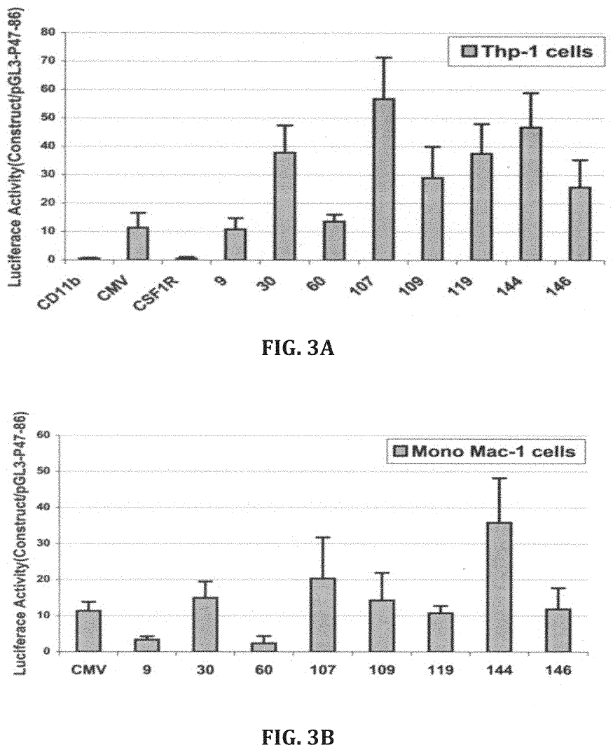

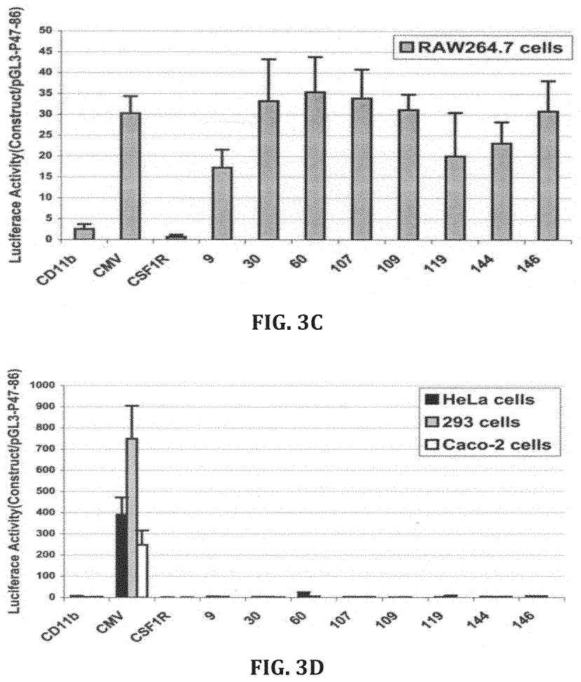

Macrophage-specific synthetic promoters. The inventors have developed a series of macrophage-specific synthetic promoter that restricts transgene expression to this lineage and characterized their strength and specificity using either a luciferase reporter assay following transient transfections in several macrophage and non-macrophage cell lines or GFP reporter in mouse models (He et al., 2006). In human monocytic cell lines Thp-1 and Mono Mac-1, and mouse macrophage cell RAW264.7 (FIG. 3) luciferase activity of the synthetic promoters was extremely high (10-200-fold over that of the CSF1R or CD11b promoters; FIG. 3). In contrast, in non-macrophage cell lines such as human intestinal epithelial cell Caco-2, cervix epithelioid carcinoma cell HeLa, embryonic kidney cell 293 (FIG. 3), T lymphocyte Jurkat, and mouse osteoblasts Oct-1 (data not shown), specific luciferase activity of the synthetic promoters was extremely low compared with the ubiquitous CMV promoter (FIG. 3).

Construction of lentiviral vectors expressing the GDNF gene driven by macrophage-specific synthetic promoter. A lentiviral vector containing the macrophage-specific synthetic promoter (see Biju et al., 2010) is based on the design described above (He et al., 2006). The macrophage-specific synthetic promoter (MSP) consists of a sequence containing two cis elements, C/EBPα and AML-1. The p47phox mini-promoter gene in the original design was replaced with a CD68 mini-promoter gene to increase specificity even further. The reporter gene (luciferase/GFP) in the original design was then replaced with a rat GDNF gene (Gene bank #NM019139, STS 50-685) using standard molecular procedures. The resulting construct was sequenced to verify the site of insertion, as well as the integrity of the GDNF gene. A similar lentiviral vector carrying the gene that encodes GFP driven by the macrophage-specific promoter was also generated and used as a control.

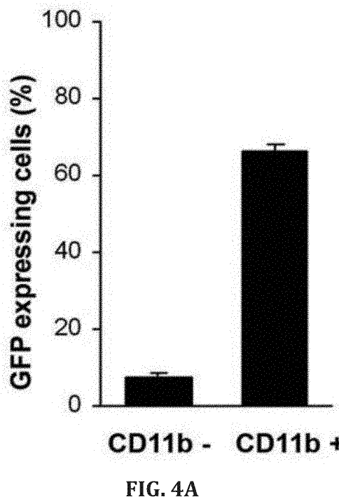

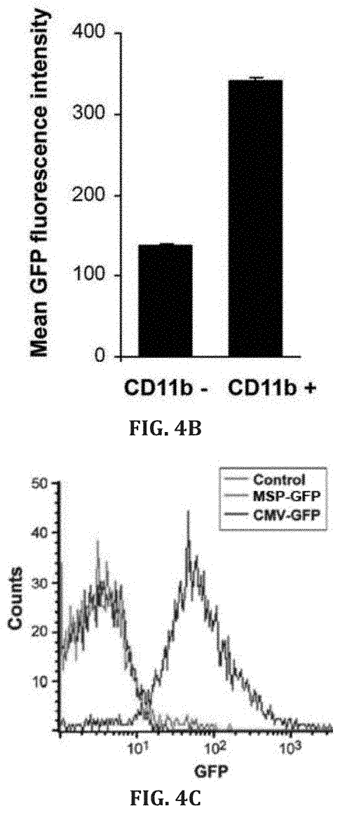

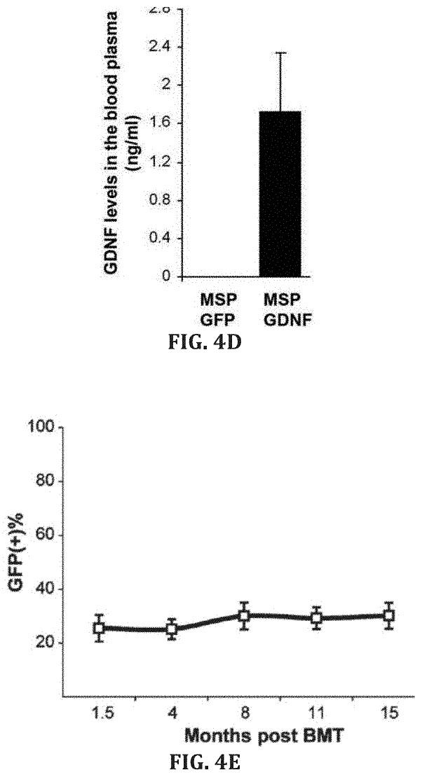

Macrophage-specific synthetic promoter drives transgene expression in monocytes /macrophages in vivo following bone marrow transplantation. Bone marrow cells from donor mice were genetically modified using lentiviral vectors encoding either GDNF or GFP driven by a macrophage-specific synthetic promoter (MSP). C57BL/6J male recipient mice seven to eight weeks of age were lethally irradiated and then transplanted with bone marrow cells transduced with either GDNF (MSP-GDNF mice) or GFP (MSP-GFP mice) vector. All transplanted animals survived without noticeable illness. After three weeks, peripheral blood samples from the recipient mice were analyzed for tissue specificity (FIG. 4A, 4B, 4C) of the synthetic promoter and its efficiency for driving synthesis and secretion of GDNF (FIG. 4D). In the MSP-GFP mice, a large proportion (approximately 66%) of the CD11b (monocyte/macrophage marker) -positive leukocytes expressed GFP (FIG. 4A), whereas only 5-7% of CD11b-negative leukocytes expressed low levels (FIG. 4A and 4B) of GFP, suggesting that the macrophage-specific synthetic promoter was driving the expression of the transgene selectively in monocytes / macrophages. No transgene expression was observed in red blood cells (FIG. 4C). In the MSP-GDNF mice a significant quantity (1.723±0.622 ng/ml) of GDNF protein was detected in the plasma (FIG. 4D), indicating that the genetically modified cells are capable of synthesizing and secreting GDNF following translation. Sustained levels of GDNF protein were detected in the plasma of MSP-GDNF mice over the entire experimental period of six months following bone marrow transplantation, whereas no GDNF was detectable in the plasma of MSP-GFP mice (FIG. 4D). To assess long-term macrophage synthetic promoter activity in vivo, a subset of seven MSP-GFP mice were used. At 1.5, 4, 8, 11, and 15 months post bone marrow transplantation peripheral blood from these mice were analyzed for GFP expression (FIG. 4E). Approximately 26-30% of the total leukocytes expressed GFP and the GFP expression was quite stable over the entire experimental period of 15 months following bone marrow transplantation.

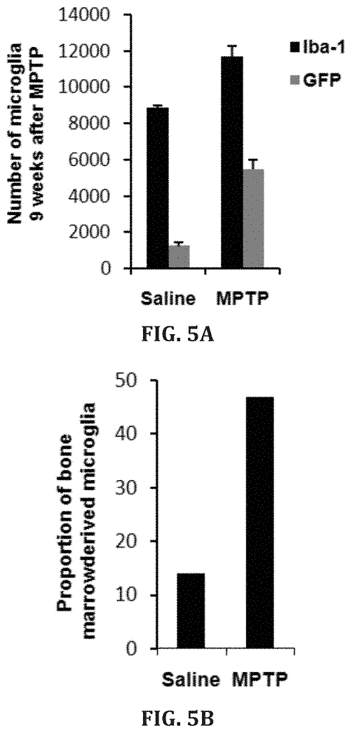

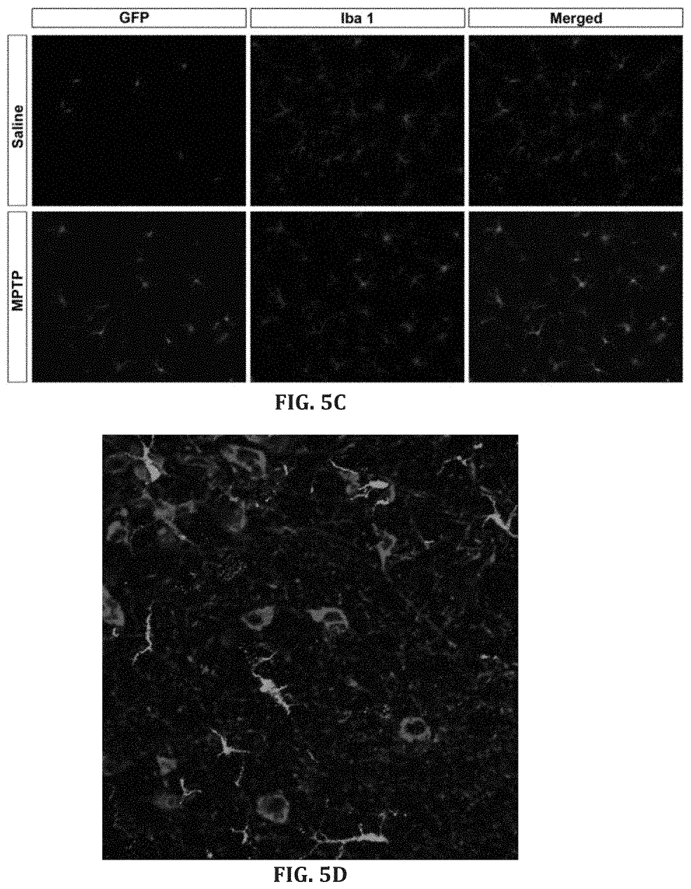

Monocytes / macrophages differentiate into microglia and their recruitment to substantia nigra is enhanced during neurodegeneration. Eight weeks after transplantation, recipient mice were injected with MPTP to induce dopaminergic neurodegeneration. MPTP dissolved in saline was injected subcutaneously into MSP-GDNF and MSP-GFP mice as follows: 15 mg/kg free base MPTP on day 1, 25 mg/kg on day 2, and 30 mg/kg on days 3-7. Control mice were treated with saline following the same regimen. Nine weeks after the last injection of MPTP or saline, MSP-GFP mice were sacrificed to evaluate the differentiation of gene-modified macrophages into microglia and their recruitment to substantia nigra. In the brain, gene-modified macrophages strongly expressed GFP, displayed the ramified morphology characteristic of microglia, and expressed Iba1, a marker for microglia. In the saline-treated MSP-GFP mice, a few GFP cells were observed in the substantia nigra, whereas the number of GFP cells was significantly increased in the substantia nigra of MPTP-treated mice (see Biju et al., 2010). In MSP-GFP mice nine weeks after the last injection of MPTP, 47% of the total microglia (Ibal-positive) in the nigra were bone marrow derived, whereas the proportion of bone marrow-derived microglia in the saline-treated MSP-GFP mice were only 14% (FIGS. 5A and 5B). In some regions of the nigra of MPTP-treated MSP-GFP mice, majority of the Ibal-positive cells were bone marrow derived (FIG. 5C). Similarly, there was a significant increase in the number of GFP positive microglia in the striatum of MSP-GFP mice following MPTP induced degeneration of dopaminergic fiber terminals (data not shown). The results indicate that genetically modified bone marrow-derived microglial cells were recruited preferentially to the site of brain insult, thus offering a proof-of-principle for the therapeutic use of bone marrow stem cell-derived macrophages for sustained delivery of GDNF to selective brain lesion sites. Most importantly, genetically modified bone-marrow derived microglia were seen in close proximity to TH-positive neurons (FIG. 5D), providing additional evidence that therapeutic molecules secreted by microglia will be accessible to dying neurons.

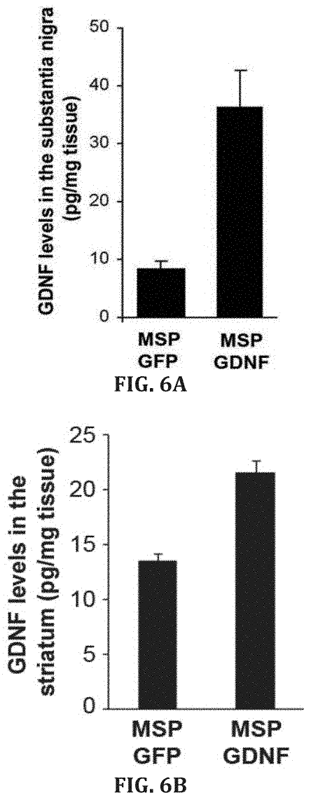

In addition, GDNF levels in the substantia nigra and striatum were measured to make certain that gene silencing did not occur following the migration of macrophages into the brain and their subsequent differentiation into microglia. In MSP-GDNF mice nine weeks after the last injection of MPTP, the mean substantia nigra GDNF protein level was 36.42±6.10 pg/mg of tissue, whereas the level of endogenous nigral GDNF in MSP-GFP mice was 8.38±1.34 pg/mg of tissue (FIG. 6A). A significant increase in the striatal GDNF level (FIG. 6B) was observed for MSP-GDNF mice (21.56±1.19 pg/mg tissue) compared with that of MSP-GFP mice (13.53±0.63 pg/mg tissue). For such a slowly progressive disease as PD, an important goal of GDNF therapy should be continuous delivery over years in order to maintain dopamine neuron survival and function. However, the broad actions of GDNF, especially on non-dopaminergic neurons, may become troublesome if a large amount of GDNF is chronically infused into the brain. Serious side-effects in clinical trials and animals experiments were attributed to very high dose of GDNF (Bohn, 1999). However, using bone marrow-derived microglia, a significant reduction in MPTP-induced neurodegeneration could be achieved with relatively low and apparently safe levels of tissue exposure to GDNF, thereby reducing dose-related side effects. Notably, brain tissue levels of GDNF in MSP-GDNF mice in the present study were about 36 pg/mg of tissue, whereas viral-mediated gene transfer resulted in up to 4200 pg/mg of tissue (Georgievska et al., 2004). In monkeys (intraventricular) and rats (nigral injection) GDNF infusion doses required for therapeutic response were between 10⁸ to 10⁹ pg (100 to 1000 μg) (Bowenkamp et al., 1995; Zhang et al., 1997). High doses (100 μg/day) of intraputamenal GDNF caused significant cerebellar Purkinje cell death (Hovland, Jr. et al., 2007).

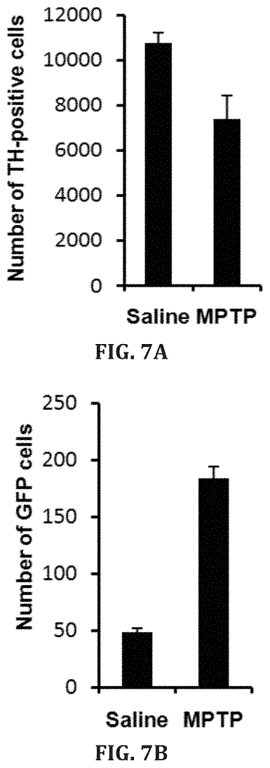

To demonstrate that the bone marrow derived microglial recruitment occurs even when the rate of neuronal death is relatively slow, MSP-GFP mice were treated with saline or MPTP using a continuous osmotic minipump infusion system (Model #2006, Alzet, Cupertion, Calif.). The minipumps were implanted subcutaneously on the upper back of the animal. The minipumps delivers saline or MPTP at a flow rate 0.35μ1/hr for 28 days. The concentration of MPTP solution was adjusted in such a way that the animals receive or 5 mg MPTP/kg daily for 28 days. On day 30 (after the start of MPTP or saline) the animals were killed and the brains were analyzed for the loss of TH-posive cells and the recruitment bone marrow derived GFP cells in the SNpc. Continuous infusion of MPTP over 28 days resulted in loss approximately 31% of TH-positive cells in the SNpc (FIG. 7A). This loss was accompanied by a four-fold increase in the number of bone marrow derived GFP cells in the SNpc (FIG. 7B), indicating that bone marrow-derived microglial recruitment occurs even when a few neurons die a day.

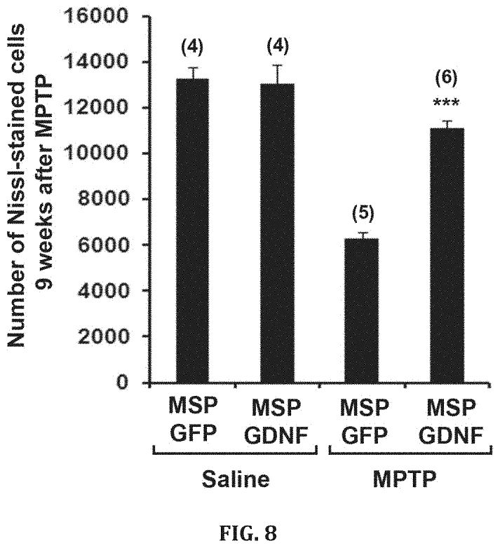

Macrophage-mediated GDNF delivery protects nigral dopaminergic neurons and their terminals in the striatum. Three or nine weeks after the last MPTP or saline injection, recipient mice were sacrificed and the neuroprotective effect of macrophage-mediated GDNF delivery on the nigrostriatal dopaminergic system was assessed by quantitative analysis of TH-positive neurons in the substantia nigra pars compacta (SNpc), as well as the density of TH-positive terminals in the striatum. The organization and intensity of TH-immunoreactive neurons were essentially similar in the saline-treated MSP-GFP and MSP-GDNF animal groups (see Biju et al., 2010). Stereological analysis demonstrated a 50-55% loss of TH-positive neurons in the SNpc of MSP-GFP mice following MPTP treatment, compared with saline-treated animals (see Biju et al., 2010). The TH-positive dendritic fiber networks in the sub stantia nigra pars reticulata (SNpr) were also dramatically reduced after MPTP treatment in MSP-GFP mice. In contrast, there was only a 15-20% MPTP-induced loss of TH-positive neurons in the SNpc of MSP-GDNF mice. Moreover, the density of the SNpr TH-positive dendritic fiber network in the MSP-GDNF mice was largely preserved in the face of MPTP treatment, relative to saline treatment. In addition, total number of Nissl-stained neurons in the SNpc was counted for each treatment group to make sure that GDNF treatment indeed prevented actual cell death (FIG. 8).

Parallel results were observed for striatal dopamine fiber terminals. In order to quantify the intensity of TH staining, optical density measurements were performed on the dorsolateral aspects of the striatum, which receive the largest share of innervation from dopamine neurons of the SNpc. By this method TH immunoreactivity within the striatum was similar between saline groups of MSP-GFP and MSP-GDNF mice (see Biju et al., 2010). Relative to the controls, there was an average 70% loss of the TH staining intensity in MSP-GFP mice sacrificed three weeks after MPTP treatment, whereas the loss in MSP-GDNF mice was only 35% (see Biju et al., 2010). Interestingly, TH staining intensity in MSP-GDNF mice improved over time. In MSP-GDNF mice sacrificed later at nine weeks after the last dose of MPTP, the reduction in the intensity of TH staining was only 15% (see Biju et al., 2010), suggesting an ongoing regenerative process within the nigrostriatal pathway. Indeed, microscopic examination of the striatum of MPTP-treated MSP-GDNF mice revealed numerous long and thick TH-positive fibers (see Biju et al., 2010) that were often branched with irregular swellings suggesting sprouting or regenerating axons of the nigral dopamine neurons. Substantially fewer fibers of this type were observed in the striatum of MPTP-treated MSP-GFP mice.

For further confirmation, tissue levels of dopamine and its metabolites, dihydroxyphenylacetic acid (DOPAC) and homovanillic acid (HVA), were determined biochemically. Compared with MPTP-treated MSP-GFP mice, the substantia nigra of MPTP-treated MSP-GDNF mice exhibited a significantly higher level of dopamine (38.8%), DOPAC (27.7%) and HVA (40.3%) (see Biju et al., 2010). Substantia nigra levels of serotonin (5-HT), another monoamine neurotransmitter, and its metabolite, 5-hydroxyindoleacetic acid (5-HIAA), were also measured to assess whether the relative preservation in levels of dopamine and its metabolites in MPTP-treated MSP-GDNF vs. MSP-GFP mice was selective or possibly a generalized effect on monoamine neurotransmitters. These analyses demonstrated similar levels of 5-HT and 5-HIAA in MPTP-treated group MSP-GFP vs. MSP-GDNF mice.

Effects of GDNF on MPTP-induced mouse behavioral impairments. General activity levels assessed by the open field test demonstrated that, relative to control mice, MPTP treatment significantly reduced the activity levels of MSP-GFP mice. In contrast, the activity of MSP-GDNF mice was preserved at levels similar to those of control mice. Moreover, MSP-GFP mice exhibited reduced food intake normalized for body weight, and this effect was reversed in MSP-GDNF mice.

Evaluation of side effects of GDNF therapy. Direct brain infusion of GDNF has been shown to cause side effects, including allodynia and weight loss (Hoane et al., 1999). In the inventors' study none of the animal showed signs of allodynia, as determined by paw withdrawal frequency or duration in response to the application of acetone on the mid-plantar surface of the hind paw. Body weight was recorded every two days throughout the duration of the experiment and expressed as mean change from initial body weight. Although both the MSP-GFP and MSP-GDNF groups lost weight acutely after whole body irradiation and transplantation, both groups regained weight quickly and then continued to gain additional weight. Over time, MSP-GFP mice gained significantly more weight than did MSP-GDNF mice, a trend that continued even after MPTP administration. GDNF exerts biological effects outside of the CNS, acting as a kidney morphogen during embryonic development and regulating the differentiation of spermatogonia in the testis. Accordingly, testes from MSP-GFP and MSP-GDNF mice were analyzed for variations possibly attributable to the differences in levels of circulating GDNF. No structural or morphological changes were observed in hematoxylin- and eosin-stained sections of testes at the light microscopic level.

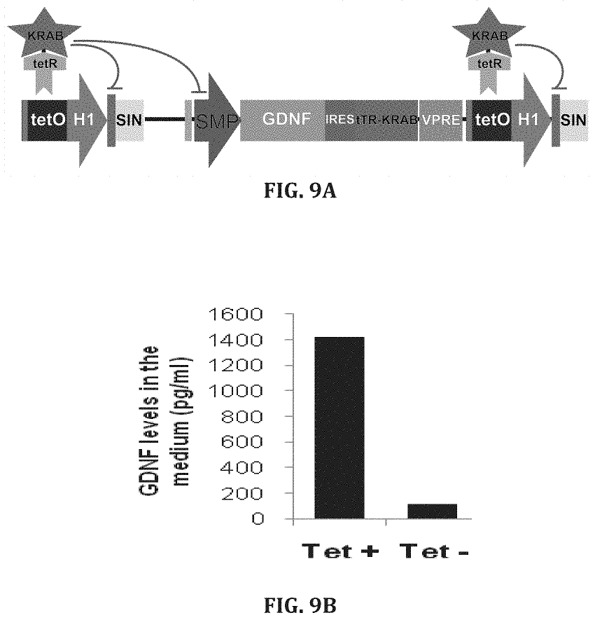

Lentiviral vector-expressing tetracycline-regulated GDNF gene driven by MSP. In the data described above, GDNF was given before MPTP-induced neurodegeneration, which would never be the case in a clinical situation, as more than 50% of the dopaminergic neurons have already been lost before detectable clinical symptoms. Therefore, it would be of much greater interest if the treatment is given after MPTP administration (restorative). Due to the complexity of the procedures involved, technically it was exceedingly difficult to do bone marrow transplantation after the start of MPTP treatment. Use of a regulated vector that can be switched on by an external factor would allow us to give GDNF at various time points after MPTP treatment and mimic early, middle and later stages of clinical parkinsonism. In this scenario, transplantation will be done before MPTP treatment; however, the expression and delivery of GDNF will be delayed until being “Switched ON” by administration of doxycycline at various time points after MPTP treatment. Toward this end the inventors developed a tetracycline regulated MSP-GDNF lentiviral vector. The latest generation of lentiviral vectors that can express therapeutic gene under the control of tetracycline administration (Szulc et al., 2006) was modified to replace PGK promoter with MSP. First, a Bsu15I site in an unessential region of lentivector pLVPT-tTR-KRAB was destroyed by partial digestion followed with blunt treatment and re-ligation. Second, the result plasmid was cut with Bsu15I at bp2148 and BamHI at bp2695 to release PGK promoter. Third, MSP was PCR amplified and inserted into the linearized lentivector to get pLVMPT-tTR-KRAB. Fourth, the plasmid was cut with BamHI at bp2695 and Smal at bp3402, to which a small linker containing BamHI-XmaI-AscI-PmeI-BsiWI-dSmaI was inserted (this step was to modify the vector in order to facilitate replacement of the EGFP gene with a therapeutic gene). Fifth, to release EGFP gene, the vector was cut with Xmal and PfI23I. The GDNF ORF was amplified by PCR and digested with AgeI and BstGI that provide compatible cohesive ends to Xmal and PfI23I, respectively. Sixth, the GDNF gene was inserted into the vector to create the final construct LV-MSP-Tet-On-GDNF(FIG. 9A). LV-MSP-Tet-On-GDNF was tested in vitro in bone marrow-derived macrophages for production of GDNF (FIG. 9B) by ELISA and shown up to 20-fold increases in GDNF protein 24 hour after addition of doxycycline (2 μg/ml).

MPTP/probenecid mouse model. Using an MPTP-only model of Parkinson's disease the inventors showed a proof-of-principle for the therapeutic use of bone marrow-derived macrophages for sustained delivery of GDNF to selective brain lesion sites. However, the MPTP-alone regimen resulted in only modest reduction (approximately 50%) in TH-positive cells. In addition, spontaneous recovery of the nigrostriatal system that is typically observed with this regimen places limits on detection of changes in motor coordination. Mice were subjected to tests for motor coordination, including rotarod test, gait test/foot print analysis, pole test, beam walking test, and grid test. None of these tests showed statistically significant differences between the saline-treated controls and the MPTP-treated group, in keeping with reports that behavioral impairment in young and adult mice exposed to MPTP occur only with very large decreases in striatal dopamine content and are often transient (Tillerson and Miller, 2003). To overcome this limit and show that bone marrow-derived macrophages mediated GDNF delivery is superior to a variety of other methods, the inventor use a chronic MPTP/probenecid (MPTP/p) mouse model. In the MPTP/p mouse model, dopamine cell loss is progressive and exceeds 70%, cytoplasmic inclusions are formed, and the behavioral impairment persists up to 6 months post-MPTP/p treatment (Meredith et al., 2008).

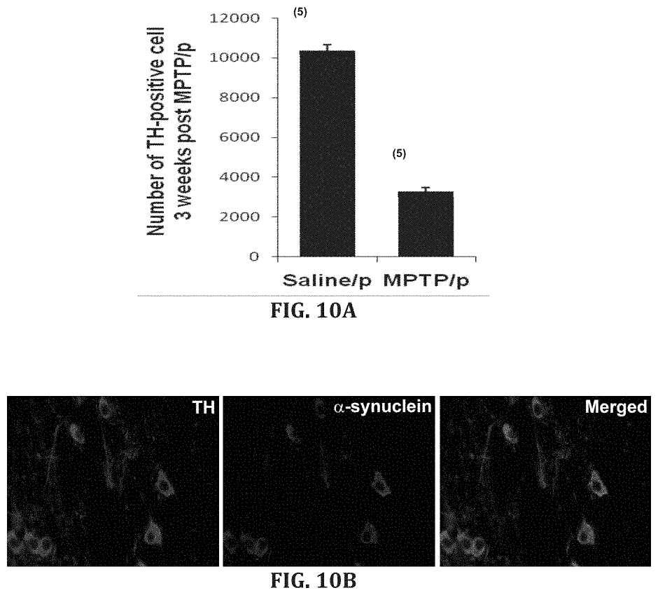

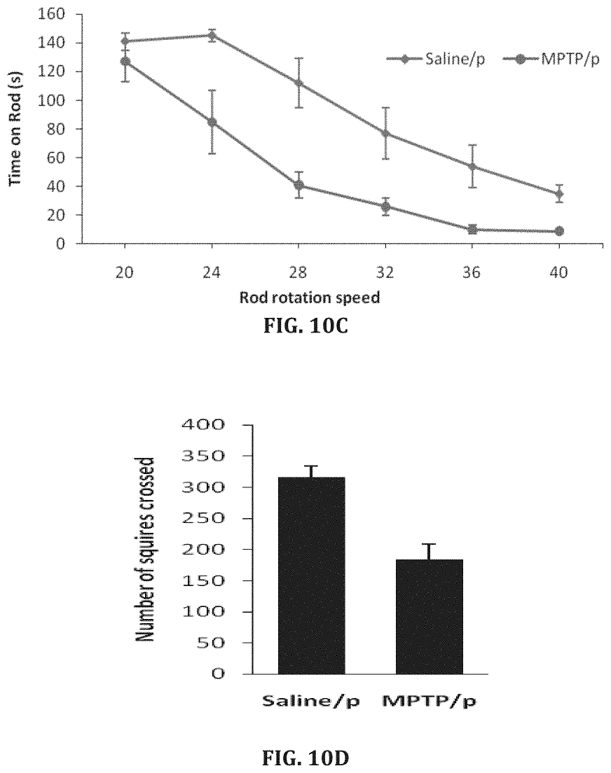

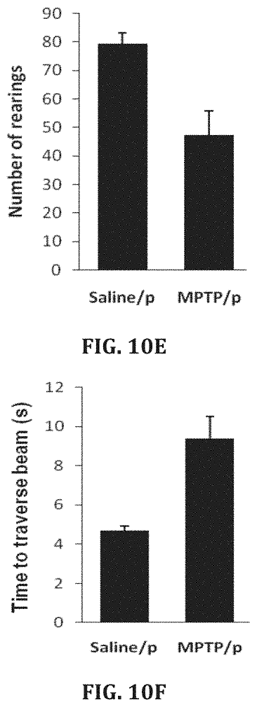

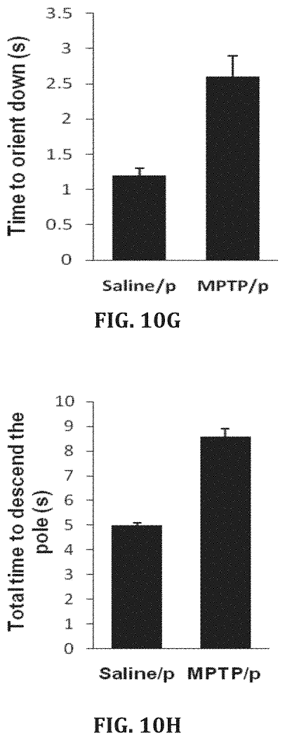

To standardize this model in the lab, eight male C57BL6/J mice weighing 20-24 g were injected with 10 doses of 25 mg/kg MPTP-HCl in saline (s.c.) and 250 mg/kg probenecid in Tris-HCl buffer (i.p.) were injected for 5 weeks at 3.5 day intervals (Meredith et al., 2008). Ten controls were similarly treated with saline and probenecid. Three weeks after MPTP/p or saline/p treatment animals were subjected to a battery of behavior tests for coordination and rigidity. Mice were then perfused transcardially with fixative (4% paraformaldehyde) and brains were processed for histology and unbiased design-based stereology. As reported the treatment resulted in approximately 70% reduction in TH-positive (FIG. 10A) neurons in the substantia nigra with many TH-positive neurons showing Lewy-like inclusions (FIG. 10B). MPTP/p treatment resulted in significant impairment in motor performance assessed by rotarod test (FIG. 10C), open field test (FIG. 10D and 10E), beam walking test (FIG. 10F) and pole test (FIG. 10G and 10H).

1. A method of non-cytotoxic stem cell transplantation in a subject for treatment of a non-cancerous condition comprising:
(a) administering at least one stem cell mobilization agent to the subject, wherein a target stem cell population migrates from host bone marrow niches into the subject's blood forming vacant bone marrow niches;
(b) removing the mobilized target stem cells from the subject, wherein competition for vacant bone marrow niches is reduced;
(c) administering genetically engineered replacement stem cells to the subject, wherein the genetically engineered replacement stem cells engraft into the vacant bone marrow niches in the subject; and
(d) repeating steps (a)-(c) two or more times;
wherein, the method does not include performing myeloablation conditioning of the subj ect.

2. The method of claim 1, further comprising removing the mobilized target stem cells by apheresis before administering genetically engineered replacement stem cells.

3. The method of claim 1, wherein the genetically engineered replacement stem cells are autologous stem cells.

4. The method of claim 1, wherein the target stem cells are hematopoietic stem cells.

5. The method of claim 1, wherein the genetically engineered replacement stem cells are hematopoietic stem cells.

6. The method of claim 1, wherein a first mobilization agent is granulocyte-colony stimulating factor.

7. The method of claim 1, further comprising administering a second mobilization agent.

8. The method of claim 7, wherein the second mobilization agent is AMD3100.

9. The method of claim 1, wherein the genetically modified replacement stem cells comprise a heterologous expression cassette.

10. The method of claim 9, wherein the expression cassette comprises a tissue specific promoter.

11. The method of claim 9, wherein the expression cassette encodes a therapeutic protein.

12. The method of claim 11, wherein the therapeutic protein is glial cell-derived neurotrophic factor (GDNF).


SEQUENCE LISTING (2 sequences)

SEQ ID NO: 1 - protein, 352 aa, Homo sapiens [sequence omitted]

SEQ ID NO: 2 - protein, 215 aa, Homo sapiens
MDYQVSSPIYDINYYTSEPCQKINVKQIAARLLPPLYSLVFIFGFVGNMLVILILINCKRLKSMTDIYLLNLAISDLFFLLTVPFWAHYAAAQWDFGNTMCQLLTGLYFIGFFSGIFFIILLTIDRYLAVVHAVFALKARTVTFGVVTSVITWVVAVFASLPGIIFTRSQKEGLHYTCSSHFPYIKDSHLGAGPAAACHGHLLLGNPKNSASVSK